(19) United States
(12) Patent Application Publication
(10) Pub. No.: US 2023/0084068 A1
(43) Pub. Date: Mar. 16, 2023

(54) DYNAMIC NONSURGICAL FACELIFT

(71) Applicant: REZA RASOULI, ANAHEIM, CA (US)

(72) Inventor: REZA RASOULI, ANAHEIM, CA (US)

(21) Appl. No.: 17/652,687

(22) Filed: Feb. 27, 2022

Related U.S. Application Data
(60) Provisional application No. 63/261,295, filed on Sep. 16, 2021.

(51) Int. Cl. A61B 17/00

(52) U.S. Cl. CPC A61B 17/00234; A61B 2017/00792; A61B 2017/00349

(57) ABSTRACT

A method of altering facial tissue comprises inserting an anchor element into an area of a scalp attached to the facial tissue; attaching a connective element to the anchor element; and using a length of the connective element to alter a position of an area of the facial tissue.

### CROSS-REFERENCE TO RELATED APPLICATIONS

This application claims priority to and the benefit of U.S. provisional patent application No. 63/261,295, filed Sep. 16, 2021, all of which is incorporated herein in its entirety.

### BACKGROUND OF THE DISCLOSURE

The present disclosure generally relates to apparatus and methods of a facelift and, more particularly, to apparatus and methods of a facelift that can be adjusted over time.

A facelift, or rhytidectomy, is a surgical procedure used to give the face a more youthful appearance. that lifts and tightens facial tissues. Surgery can involve the removal of excess facial skin and the tightening of underlying tissues.

A traditional facelift can involve an incision in front of the ear and extending into the hairline. The incision can curve around the bottom of the ear and then behind the ear and to the hairline at the back of the neck. The skin can then be separated from deeper tissues. The deeper tissues can then be tightened with sutures with or without the removal of deeper tissues. The skin can then be re-draped and excess skin removed. The traditional facelift presents the typical risks of surgery.

A thread facelift is non-surgical alternative to a traditional facelift. A suture or thread is inserted into the facial skin and pulled tight. The thread then dissolves over time, though the effects of the lift are intended to continue. The thread lift tends to lose beneficial effects sooner than the traditional facelift.

As can be seen, there is a need for improved apparatus and methods of altering facial tissue, such as for a facelift, which is minimally invasive and can be dynamically adjusted over time.

### SUMMARY OF THE DISCLOSURE

In one aspect of the present disclosure, a method of altering facial tissue comprises attaching a connective element to areas of a scalp connected to the facial tissue; and adjusting a length of the connective element to pull the facial tissue towards the scalp.

In another aspect of the present disclosure, a method of altering facial tissue comprises inserting an anchor element into an area of a scalp attached to the facial tissue; attaching a connective element to the anchor element; and using a length of the connective element to alter a position of an area of the facial tissue.

In a further aspect of the present disclosure, a kit for altering facial tissue comprises an anchor element configured to be inserted into a scalp connected to the facial tissue and further configured to open and close; an attaching element configured to releasable connect to the anchor element; and a connective element configured to attach to the attaching element.

These and other features, aspects and advantages of the present disclosure will become better understood with reference to the following drawings, description, and claims.

### BRIEF DESCRIPTION OF THE DRAWINGS

FIGS. 1A-1C are planar views of a facelift assembly in use according to an embodiment of the present disclosure.

FIG. 2 is a perspective view of an anchor element of a facelift assembly according to another embodiment of the present disclosure.

FIG. 3 perspective view of an attaching element of a facelift assembly according to an embodiment of the present disclosure.

FIG. 4 is a is a planar view of an attaching element/connective element of a facelift assembly according to an embodiment of the present disclosure.

FIG. 5 is a side view of a padded connective element of a facelift assembly according to an embodiment of the present disclosure.

FIG. 6 is a planar view of a facelift assembly in use according to another embodiment of the present disclosure.

FIGS. 7A-7F are views of a method according to an embodiment of the present disclosure.

### DETAILED DESCRIPTION OF THE DISCLOSURE

The following detailed description is of the best currently contemplated modes of carrying out the disclosure. The description is not to be taken in a limiting sense, but it is merely for the purpose of illustrating the general principles of the disclosure, since the scope of the disclosure is best defined by the appended claims.

Various inventive features are described below that can each be used independently of one another or in combination with other features. However, any single inventive feature may not address any of the problems discussed above or may only address one of the problems discussed above. Further, one or more of the problems discussed above may not be fully addressed by any of the features described below.

As used herein, the terms “embodiment” and “embodiments” are intended to be used interchangeably. In other words, the singular includes the plural, and vice versa.

As used herein, the term “facelift” is intended to mean “rhytidectomy”, as well as alterations of facial tissue in general.

Here, the technical problems to be solved are that a facelift involving surgery has attendant risks. Also, a face continues to change over time following a facelift. Those changes can alter the initial results of the facelift, and the changes may be undesirable.

Broadly, the present disclosure solves the foregoing problems by apparatus and methods for a nonsurgical facelift—i.e., altering facial tissue. The present disclosure does not require general anesthesia. The present disclosure further solves the foregoing problems by apparatus and methods for a facelift that can dynamically adjust to changing facial tissue over time. The present disclosure may lift facial tissue on opposite sides of the face or just one side of the face.

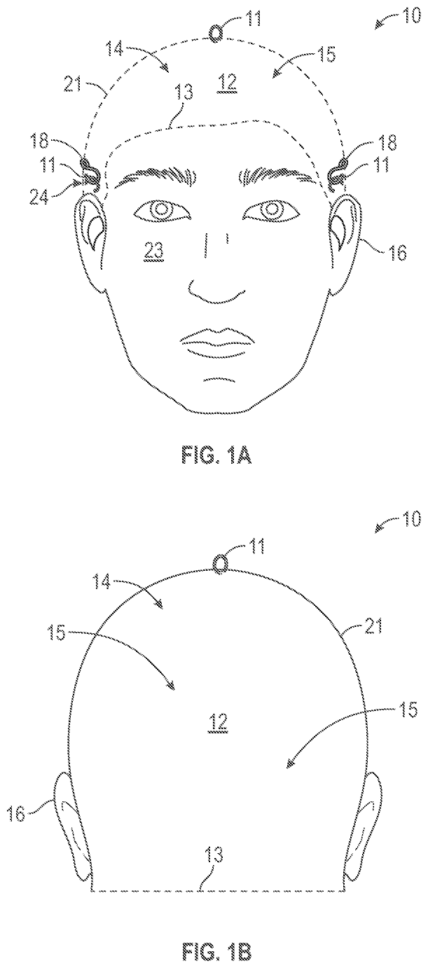

FIGS. 1A-1C are front, back, and side views of a facelift assembly 10 (i.e., kit) in use on a patient according to an embodiment of the present disclosure. The assembly or kit 10 may include one or more anchor elements 11. One or more of the anchor elements 11 may be configured to be inserted into a scalp 12 of the patient, according to an embodiment. In an embodiment, the one or more anchor elements 11 may be configured to be inserted into one or more different areas of the scalp 12. The different scalp areas may include, for example, a top area 14, an area 15 beyond a hairline 13 (i.e., between the hairline and the top area), and/or an area 24 adjacent to an ear 16 of the patient, in embodiments. The hairline 13 may be a border between the scalp and skin (e.g., forehead or neck).

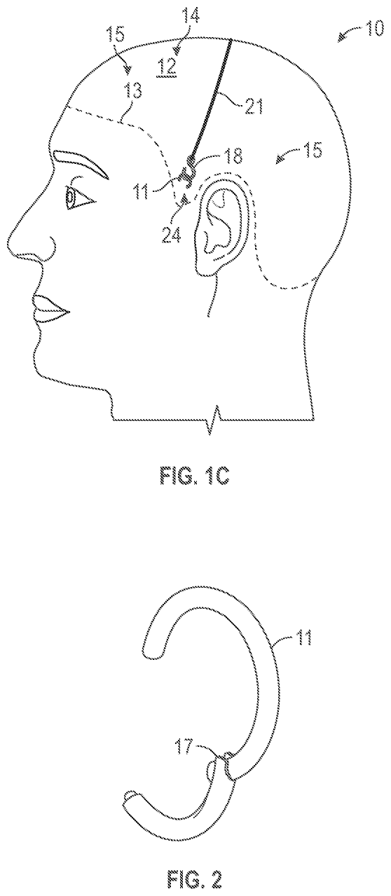

In FIG. 2, according to an embodiment, one or more of the anchor elements 11 can be configured as a ring. The anchor element(s) 11 can include a hinge 17. One or more of the anchor elements 11 may be configured to open and close. In an embodiment, when closed, the anchor element(s) 11 may be configured to lock the anchor element(s) from opening.

In an embodiment, the anchor element(s) 11 may be made of a material that is non-reactive with human tissue. In an embodiment, the anchor element(s) 11 may be made of a metal, such as titanium. According to an embodiment, the anchor element(s) 11 may have a diameter of about 5 mm and a thickness (i.e., a height measured in a direction perpendicular to the diameter) of about 0.8 mm. However, the present disclosure envisions other diameters and thicknesses.

In an embodiment, one or more of the anchor elements 11 may be colored. The color of the anchor element(s) 11 may match a color of hair on the scalp 12, according to an embodiment.

Referring again to FIGS. 1A-1C, the facelift assembly or kit 10 may include one or more attaching elements 18, according to an embodiment. One or more of the attaching elements 18 may be configured to releasably connect to one or more of the anchor elements 11, according to an embodiment. One of more of the attaching elements 18 may be configured to connect to one or more of the anchor elements 11 when the anchor element(s) is open or closed. The attaching element(s) 18 may be configured to pass through the anchor element(s) when closed.

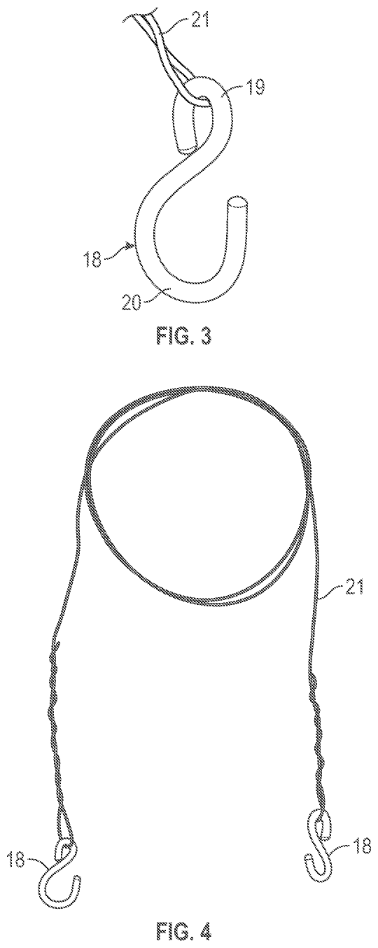

In FIG. 3, according to an embodiment, the attaching element(s) 18 may be a hook. In an embodiment, the attaching element(s) 18 may have a FIG. 8 configuration. In an embodiment, the attaching element(s) 18 may have a closed loop 19 an open loop 20.

According to an embodiment, the open loop 20 can be configured to releasably connect to an anchor element(s) 11. In an embodiment, the open loop 20 may have a diameter larger than that of the closed loop 19. A diameter of the open loop 20 may be smaller than a diameter of the anchor element(s) 11, in an embodiment. The attaching element(s) 18, such as the closed loop 19, can be configured to releasably connect to a connective element 21 described below.

In an embodiment, the attaching element(s) 18 may be made of a material that is non-reactive with human tissue. In an embodiment, the attaching element(s) 18 may be made of a metal, such as titanium. According to an embodiment, the attaching element(s) 18 may have a thickness of about 0.8 mm. In an embodiment, the open loop 20 may have a diameter of about 3-4 mm with an opening of about 0.9-1 mm. However, the present disclosure envisions other diameters and thicknesses.

Referring again to FIGS. 1A-1C, the facelift assembly or kit 10 may include one or more connective elements 21, according to an embodiment. In an embodiment, the connective element(s) 21 may be a flexible, non-stretchable material. According to an embodiment, the one or more connective element(s) 21 can be one of a wire, a cable, a thread, a cord, and a string. In an embodiment, the connective element(s) 21 is titanium wire #28. However, other dimensions are envisioned.

In an embodiment, one or more connective elements 21 may be configured to attach to opposed lateral sides or just one side of the scalp 12. In embodiments, one or more connective elements 21 may have one or two distal ends configured to releasably attach to the anchor element(s) 11 and/or to the attaching element(s) 18. In another embodiment, one or more of the connective elements 21 may have one or both ends that is/are, in fact, attached to the anchor element(s) 11 and/or to the attaching element(s) 18.

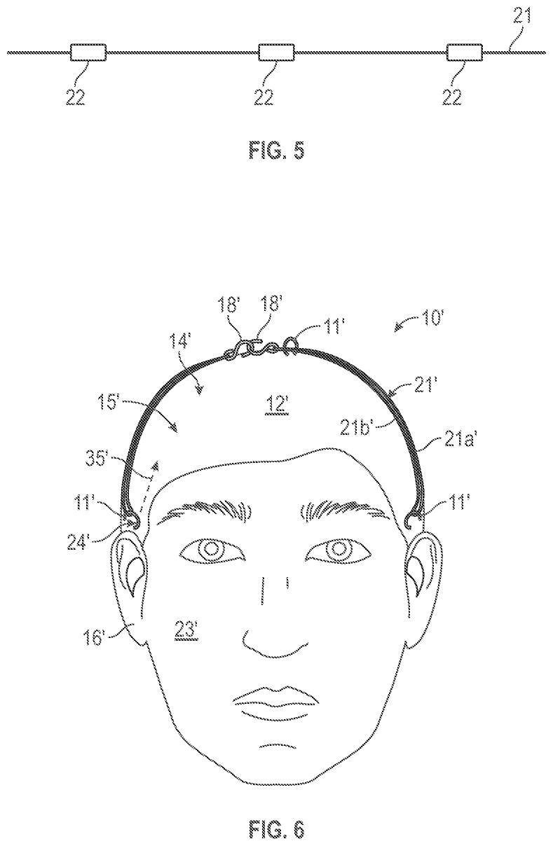

In FIG. 4, in an embodiment, one or both ends of the connective element(s) 21 are either configured to attach or are in fact attached to the closed loop 19 of the attaching element(s) 18. According to an embodiment, one or both distal ends of the connective element(s) 21 may be configured to be inserted into the closed loop 19 and that distal end(s) may be configured to wrap around a length of the connective element(s) 21 that leads into the closed loop 19.

In an embodiment, one or both ends of the connective element(s) 21 may be configured to be inserted in the anchor element 11 and that end(s) may be configured to wrap around a length of the connective element(s) 21 that leads into the anchor element 11.

Thus, in an embodiment, the portion of the connective element(s) 21 that has been wrapped or twisted around the length of the connective element(s) that leads into either the anchor element 11 or attaching element 18 may be unwrapped or untwisted, according to an embodiment.

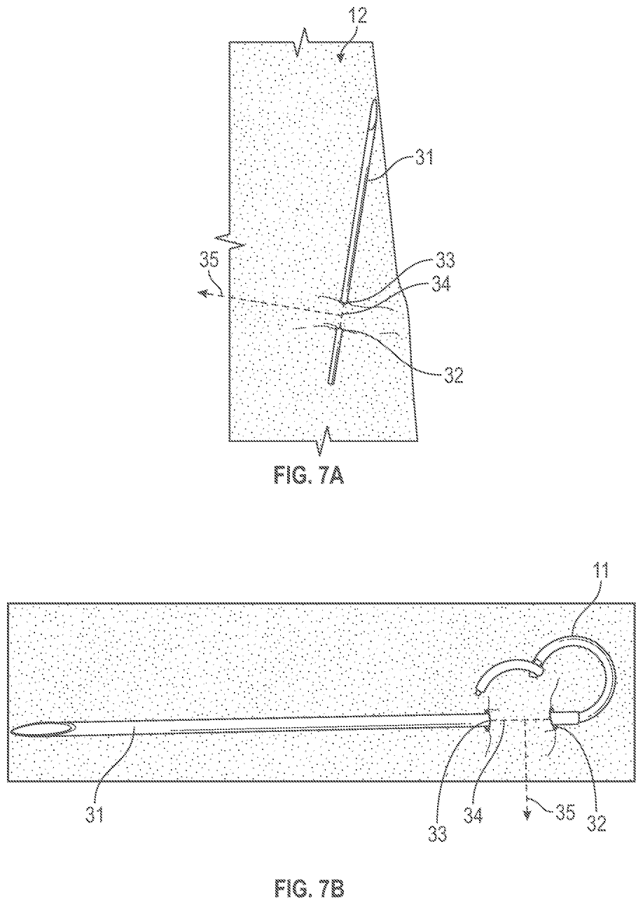

FIG. 5 depicts a connective element 21 having one or more pads 22, in an embodiment. The pad(s) 22 may be configured to cushion contact between the connective element 21 and the scalp, in an embodiment. Therefore, in an embodiment, the pad(s) 22 may be made of a material such as a gel or foam.

In an embodiment, the facelift assembly or kit 10 may include a guide needle 31 described below, in an embodiment. The guide needle 31 may be configured to puncture the scalp 12, in an embodiment.

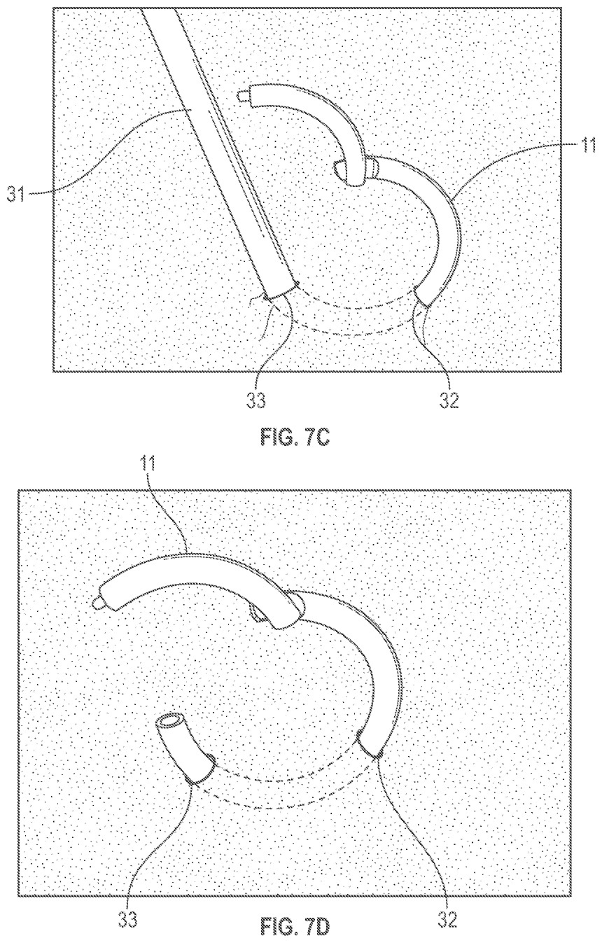

FIG. 6 depicts another embodiment of a facelift assembly or kit 10′. In an embodiment, an anchor element 11′ can be configured to be inserted into an area(s) of the scalp 12′, such as at opposite sides and top. A connective element 21′ can be configured to loop through the anchor elements 11′ at the sides of the scalp 12′, in an embodiment. Thereby, the connective element 21′ may be configured to be split into a first part 21a′ and a second part 21b′, in an embodiment. The first and second parts 21a′, 21b′ may be configured to extend generally parallel to one another over the scalp 12′.

According to an embodiment, the first and second parts 21a′, 21b′ may be configured to extend through an anchor element 11′ at a top of the scalp 12′. In an embodiment, distal ends of the connective element 21′ may be attached to a respective attaching element 18′. In an embodiment, the respective attaching elements 18′ may be configured to attach to one another at the top of the scalp, or in other areas of the scalp.

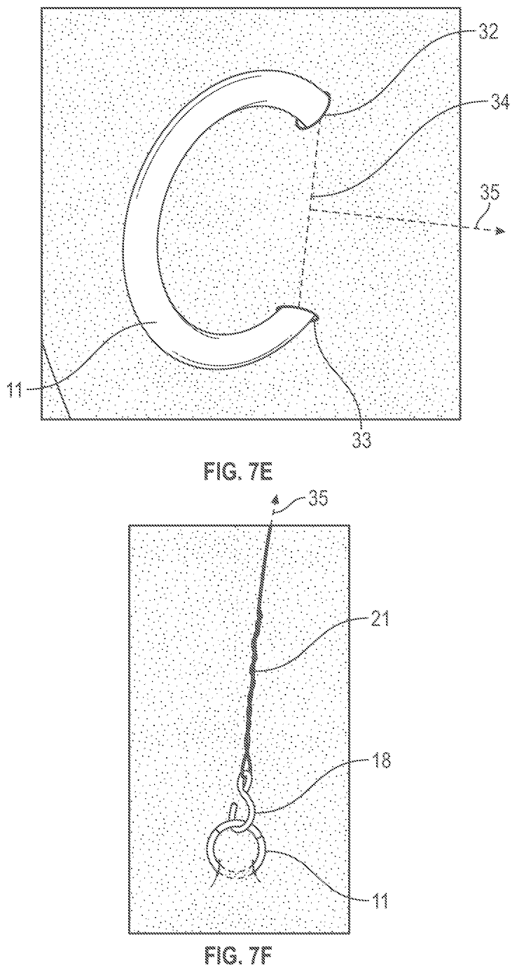

FIGS. 7A-7E depict a method 30 according to an embodiment of the present disclosure.

In FIG. 7A, the method 30 may include cleaning and/or disinfecting an area of the scalp 12 where an anchor element 11 is to be inserted, as well as the components in the kit 10, in an embodiment. A local anesthesia may also be used.

The method 30 may include creating an entry point (hole) 32 and an exit point (hole) 33 in the scalp 12, in an embodiment. For example, a pair of entry/exit points 32, 33 may be at a top area 14 of the scalp 12. In another example, a pair of entry/exist points 32, 33 may be at an area 15 beyond a hairline 13 (e.g., between the hairline 13 and the top area 14 of the scalp 12, between the hairline and a side area of the scalp, between the hairline and a back area of the scalp, or between the hairline and a front area of the scalp).

According to an embodiment, the method 30 may include creating two or more pairs of entry/exit points 32, 33 in two or more areas of the scalp 12, according to an embodiment. For example, the method 30 can include creating a respective pair of entry/exit points 32, 33 at opposite lateral sides of the scalp 12. In another example, the method 30 can include creating a respective pair of entry/exit points 32, 33 at areas adjacent to both ears 16. In a further example, the method 30 may include creating three pairs of entry/exit points 32, 33—at a top area 14 of the scalp 12 and at opposite lateral sides of the scalp 12 (FIGS. 1A and 6).

According to an embodiment, the method 30 may include using a guide needle 31 to pierce/puncture the scalp 12 to create one or more entry and/or exit points 32, 33.

In FIG. 7A, the method 30 may include correlating a distance between the entry point 32 and the exit point 33 to a diameter of the anchor element 11 to be used at the entry/exit points 32, 33, in an embodiment. Accordingly, in an embodiment, a distance between the entry point 32 and the exit point 33 may be the same or about the same as a diameter of the anchor element 11.

In an embodiment, the method 30 may include creating an entry/exit line 34 between the entry point 32 and the exit point 33. The entry/exit line 34 may, in an embodiment, be a straight line.

In an embodiment, the method 30 can include orienting the entry/exit line 34 to a lifting vector 35. In an embodiment, the lifting vector 35 may be parallel to a direction of, or along a line of, desired lifting (i.e., pulling) of the facial tissue 23. In an embodiment, the lifting vector 35 may represent a direction and magnitude that the connective element 21 may pull on the anchor element 11 described below. The orientation between the entry/exit line 34 and lifting vector 35 can be perpendicular or nearly perpendicular, in an embodiment. However, other orientations are envisioned.

In FIG. 7B, according to an embodiment, the method 30 may include leaving an exposed distal end of the guide needle 31 outside of the entry point 32. The method 30 may include attaching an open end of an anchor element 11 to the exposed distal end of the guide needle 31, according to an embodiment. The method 30 may include removing the guide needle 31 from the entry point 32. In an embodiment, the method 30 may include inserting the open end of the anchor element 11 into the entry point 32. In an embodiment, the method 30 can include inserting the anchor element 11 in the skin of the scalp 12. In an embodiment. the method 30 may further include concurrently leaving the guide needle 31 in the exit point 33.

In an embodiment, the method 30 may further include moving the open end of the anchor element 11, while attached to the guide needle 11, in the skin of the scalp and towards the exit point 33. In an embodiment, the method 30 may include using a hemostat to move the open end of the anchor element 11 and guide needle 30.

In FIG. 7C, the method 30 may include moving the open end of the anchor element 11, while attached to the guide needle 11, to and/or out of the exit point 33.

In FIG. 7D, the method 30 may include detaching the guide needle 31 from the anchor element 11, in an embodiment. The method 30 may include removing the guide needle 31 from the exit point 33, in an embodiment. In an embodiment, the method 30 may include positioning a part (e.g., half) of the anchor element 11 in the scalp and positioning another part (e.g., the other half) of the anchor element 11 out of the scalp. In an embodiment, the part of the anchor element 11 in the scalp can extend into the skin of the scalp but not below the same.

In FIG. 7E, the method 30 may include closing the ends of the anchor element 11, in an embodiment. The method 30 may also include locking the ends of the anchor element 11, in an embodiment. For example, the anchor element 11 may be configured to enable self-locking. In another example, the ends and/or the hinge 17 of the anchor element 11 may be glued together.

In FIG. 7F, the method 30 may include attaching the connective element(s) 21 to one or more areas of the scalp 12, such as the top area 14, the area 15 beyond the hairline 13, and/or the area 24 adjacent the ear 16, in an embodiment. The method 30 may include attaching one or more connective elements 21 to opposed lateral sides of the scalp 12, in an embodiment.

In attaching the connective element(s) to the scalp, the method 30 may include positioning a first anchor element 11 (of three anchor elements 11) according to a first and/or a second lifting vector 35, in an embodiment. For example, the first anchor element 11 may be positioned at the top area 14 of the scalp such as when the facial tissue is to be lifted towards the top of the scalp. The method 30 may then include passing a first end of one connective element 21, having a first attaching element 18 thereat, through the first anchor element 11, in an embodiment. (FIG. 1A). Thereby, a second end of the one connective element 21, having a second attaching element 18 thereat, can be on a side of the first anchor element 11 opposite the first end of the connective element 21. In the foregoing embodiment, the one connective element 21 may freely move back and forth through the first anchor element 11.

According to the method 30, in an embodiment, a second anchor element 11 may be positioned according to the first lifting vector 35. For example, if the top area 14 of the scalp is a terminal point of the first lifting vector 35, the area 24 adjacent the ear 16 can be an originating point of the first lifting vector 35. Thus, in the foregoing example, the second anchor element 11 may be positioned in the area 24 adjacent the ear 16. (FIGS. 1A, 1C).

In the method 30, the first attaching element 18 may then be attached/connected to the second anchor element 11, in an embodiment. In an embodiment, the first attaching element 18 may be releasably attached/connected to the second anchor element 11. The open loop 20 of the first attaching element 18 may hook the second anchor element 11, in an embodiment. (FIGS. 1A, 1C).

According to the method 30, in an embodiment, a third anchor element 11 may be positioned according to a second lifting vector 35. In embodiments, the first and second lifting vectors 35 may be the same or different. For example, if the top area 14 of the scalp is a terminal point of the second lifting vector 35, the area 24 adjacent the other ear 16 can be an originating point of the second lifting vector 35. Thus, in the foregoing example, the third anchor element 11 may be positioned in the area 24 adjacent the other ear 16. (FIGS. 1A, 1C).

In the method 30, the second attaching element 18 may then be attached/connected to the third anchor element 11, in an embodiment. In an embodiment, the second attaching element 18 may be releasably attached/connected to the third anchor element 11. The open loop 20 of the second attaching element 18 may hook the third anchor element 11, in an embodiment. (FIGS. 1A, 1C).

In another embodiment of the method 30, the third anchor element 11 above can be omitted. For example, the omission of the third anchor element 11 may occur when only one side of the face is to be lifted. According to an embodiment, with the omission of the third anchor element 11, the one connective element 21 may be attached to the first and second anchor elements 11, such as by the first and second attaching elements 18.

In the method 30, the order of positioning and attaching the anchor elements 11 and the attaching elements 18 may vary, according to embodiments.

In a method 30′, according to an embodiment, there can be a positioning of a first anchor element 11′ (of three anchor elements 11′) according to a first and/or a second lifting vector 35′, in an embodiment. For example, the first anchor element 11′ may be positioned at the top area 14′ of the scalp such as when the facial tissue is to be lifted towards the top of the scalp. The method 30′ may then include passing a first end of one connective element 21′, having a first attaching element 18′ thereat, through the first anchor element 11′, in an embodiment. (FIG. 6). Thereby, a second end of the one connective element 21′, having a second attaching element 18′ thereat, can be on a side of the first anchor element 11′ opposite the first end of the connective element 21′. In the foregoing embodiment, the one connective element 21′ may freely move back and forth through the first anchor element 11′.

According to the method 30′, in an embodiment, a second anchor element 11′ may be positioned according to the first lifting vector 35′. For example, if the top area 14′ of the scalp is a terminal point of the first lifting vector 35′, the area 24′ adjacent the ear 16′ can be an originating point of the first lifting vector 35′. Thus, in the foregoing example, the second anchor element 11′ may be positioned in the area 24′ adjacent the ear 16′. (FIG. 6). The first attaching element 18′ may then be passed through, as opposed to attached to, the second anchor element 11′, in an embodiment.

According to the method 30′, in an embodiment, a third anchor element 11′ may be positioned according to a second lifting vector 35′. In embodiments, the first and second lifting vectors 35′ may be the same or different. For example, if the top area 14′ of the scalp is a terminal point of the second lifting vector 35′, the area 24′ adjacent the other ear 16′ can be an originating point of the second lifting vector 35′. Thus, in the foregoing example, the third anchor element 11′ may be positioned in the area 24′ adjacent the other ear 16′. (FIG. 6). The second attaching element 18 may then be passed through, as opposed to attached to, the third anchor element 11, in an embodiment.

Thereafter, in an embodiment, the method 30′ can include connecting the two attaching elements 18′ together, in an area of the scalp 12′, such as at a top area 14′ of the scalp 12 (FIG. 6).

In another embodiment of the method 30′, the third anchor element 11′ above can be omitted. For example, the omission of the third anchor element 11′ may occur when only one side of the face is to be lifted. According to an embodiment, with the omission of the third anchor element 11′, the one connective element 21′ may be attached to the first anchor element 11′ (and not the second anchor element 11′), such as by the first and second attaching elements 18′.

In the method 30′, the order of positioning and attaching the anchor elements 11′ and the attaching elements 18′ may vary, according to embodiments.

Referring again to FIG. 7E, the method 30/30′ can include releasably connecting the connective element(s) 21/21′ with the attaching element(s) 18/18′, in an embodiment. The method 30/30′ can include inserting an end of the connective element (s) 21/21′ through the attaching element(s) 18/18′, such as through the closed loop(s) 19/19′. The method 30/30′ can include twisting or wrapping the end of the connective element(s) 21/21′ around a portion of the connective element(s) 21/21′.

In an embodiment, the method 30/31′ can include altering or adjusting a length of the connective element(s) 21/21′. The method 30/30′ can include using the length of the connective element(s) 21/21′ to alter (e.g., lift) a position of an area of the facial tissue 23/23′, according to an embodiment. The method 30/31′ can include altering or adjusting the length of the connective element(s) 21/21′ to pull the facial tissue 23/23′ towards an area of the scalp 12/12′, according to an embodiment. The area of the scalp 12/12′ may be the top area 14/14′, the area 15/15′ beyond the hairline 13/13′, and/or an area 24/24′ adjacent the ear 16/16′, for example.

Concurrently with altering or adjusting the length of the connective element 21/21′, the method 30/30′ may include manually altering (e.g., lifting) a position of an area of the facial tissue 23/23′, in an embodiment. For example, a user's hand may lift the area of facial tissue.

The method 30/30′ can include changing a length of the connective element(s) 21/21′, such as shortening the length, in an embodiment. For example, in an embodiment, changing the length of the connective elements 21/21′ may include changing the amount of the connective element(s) 21/21′ that is twisted or wrapped around itself. In another example, changing the length of the connective element(s) 21/21′ may include replacing the existing connective element(s) 21/21′ with another connective element(s) 21/21′ of a different length.

Concurrently with changing a length of the connective element(s) 21/21′, the method 30/30′ may include manually altering (e.g., lifting) a position of an area of the facial tissue 23/23′, in an embodiment.

The method 30/30′ can include pulling the connective element(s) 21/21′ in the vector direction 35/35′ and thereby lifting the facial tissue 23/23′ in the vector direction 35/35′, in an embodiment.

In an embodiment, all or parts of the method 30/30′ may be repeated over time or in a plurality of treatment sessions. In an embodiment, after the anchor element(s) 11/11′ has been inserted into the scalp 12/12′, the connective element(s) 21/21′ may be de-attached from one or more of the attaching elements 18/18′ and/or one or more of the anchor elements 11/11′. Upon detachment, the length of the connective element(s) 21/21′ may be adjusted, such as shortened, in an embodiment. The shortening may be implemented by increasing the amount of the connective element 21/21′ that is twisted/wrapped around itself, in an embodiment. After the length is adjusted, the connective element(s) 21/21′ may be re-attached to the attaching element(s) 18/18′ or to the anchor element(s) 11/11′, in an embodiment.

It can be appreciated that the method 30/30′ of the above detaching, length adjusting, and re-attaching can occur over time, such as over a period of months or years, in an embodiment. Also, the above detaching, length adjusting, and re-attaching can occur in a plurality of treatment sessions, in an embodiment. In so doing, the altering of facial tissue can change over time as the facial tissue changes over time.

It should be understood, of course, that the foregoing relates to exemplary embodiments of the disclosure and that modifications may be made without departing from the spirit and scope of the disclosure as set forth in the following claims.

1. A method of altering facial tissue, comprising:
attaching a connective element to areas of a scalp connected to the facial tissue; and
adjusting a length of the connective element to pull the facial tissue towards the scalp.

2. The method of claim 1, wherein the connective element is selected from a group consisting of a wire, a cable, a thread, a cord, and a string.

3. The method of claim 1, wherein attaching the connective element includes attaching the connective element to an area beyond a hairline.

4. The method of claim 1, wherein attaching the connective element occurs periodically over time.

5. The method of claim 1, wherein attaching the connective element occurs in a plurality of treatment sessions.

6. The method of claim 1, wherein adjusting the length of the connective element occurs periodically over time.

7. The method of claim 1, wherein adjusting the length of the connective element occurs in a plurality of treatment sessions.

8. A method of altering facial tissue, comprising:
inserting an anchor element into an area of a scalp attached to the facial tissue;
attaching a connective element to the anchor element; and
using a length of the connective element to alter a position of an area of the facial tissue.

9. The method of claim 8, further comprising inserting another anchor element in another area of the scalp.

10. The method of claim 9, wherein:
the area of the scalp and the another area of the scalp are on opposite sides of the scalp.

11. The method of claim 9, wherein:
the area of the scalp is one of a side of the scalp and a top of the scalp; and
the another area of the scalp is the other of the side of the scalp and the top of the scalp.

12. The method of claim 8, wherein the anchor element is a ring.

13. The method of claim 8, wherein the connective element is a wire.

14. A kit for altering facial tissue, comprising:
an anchor element configured to be inserted into a scalp connected to the facial tissue and further configured to open and close;
an attaching element configured to releasable connect to the anchor element; and
a connective element configured to attach to the attaching element.

15. The kit of claim 14, further comprising two anchor elements.

16. The kit of claim 14, further comprising two attaching elements.

17. The kit of claim 14, wherein the connective element is attached to the attaching element.

18. The kit of claim 14, further comprising a pad configured to attach to the connective element.

19. The kit of claim 14, further comprising a pad attached to the connective element.

20. The kit of claim 14, further comprising a guide needle configured to puncture the scalp.
